(19) United States
(12) Patent Application Publication
(10) Pub. No.: US 2007/0209668 A1
(43) Pub. Date: Sep. 13, 2007

(54) Combination Pressure Therapy for Treatment of Spinal Cord Injury, Intervertebral Disc Hydration, Inflammation, & Wound Healing

(75) Inventor: Carl Linton, Temecula, CA (US)

Correspondence Address: WILSON SONSINI GOODRICH & ROSATI, 650 PAGE MILL ROAD, PALO ALTO, CA, 94304-1050, US

(73) Assignee: CVAC SYSTEMS, INC., Temecula, CA (US)

(21) Appl. No.: 11/672,937

(22) Filed: Feb. 8, 2007

Related U.S. Application Data
(60) Provisional application No. 60/771,848, filed on Feb. 8, 2006.
(60) Provisional application No. 60/772,647, filed on Feb. 10, 2006.
(60) Provisional application No. 60/773,460, filed on Feb. 15, 2006.
(60) Provisional application No. 60/773,585, filed on Feb. 15, 2006.
(60) Provisional application No. 60/774,441, filed on Feb. 17, 2006.
(60) Provisional application No. 60/775,917, filed on Feb. 22, 2006.
(60) Provisional application No. 60/775,521, filed on Feb. 21, 2006.
(60) Provisional application No. 60/745,723, filed on Apr. 26, 2006.
(60) Provisional application No. 60/824,890, filed on Sep. 7, 2006.
(60) Provisional application No. 60/822,375, filed on Aug. 14, 2006.
(60) Provisional application No. 60/826,061, filed on Sep. 18, 2006.
(60) Provisional application No. 60/826,068, filed on Sep. 18, 2006.
(60) Provisional application No. 60/743,470, filed on Mar. 13, 2006.
(60) Provisional application No. 60/745,721, filed on Apr. 26, 2006.

(51) Int. Cl. A61B 19/00

(52) U.S. Cl. 128/898

(57) ABSTRACT

Methods for administering pressure changes to a user for the treatment and prevention of diseases and conditions are disclosed herein. Methods of administering Cyclic Variations in Altitude Conditioning Sessions (CVAC Session(s)) for the treatment of spinal cord injury, intervertebral disc therapy, inflammation, and wound healing are disclosed herein.

### CROSS-REFERENCE

This application claims the benefit of U.S. Provisional Application No. 60/771,848, filed Feb. 8, 2006, U.S. Provisional Application No. 60/772,647, filed Feb. 10, 2006, U.S. Provisional Application No. 60/773,460, filed Feb. 15, 2006, U.S. Provisional Application No. 60/773,585, filed Feb. 15, 2006, U.S. Provisional Application No. 60/774,441, filed Feb. 17, 2006, U.S. Provisional Application No. 60/775,917, filed Feb. 22, 2006, U.S. Provisional Application No. 60/775,521, filed Feb. 21, 2006, U.S. Provisional Application No. 60/743,470, filed Mar. 13, 2006, U.S. Provisional Application No. 60/745,721, filed Apr. 26, 2006, U.S. Provisional Application No. 60/745,723, filed Apr. 26, 2006, U.S. Provisional Application No. 60/824,890, filed Sep. 7, 2006, U.S. Provisional Application No. 60/822,375, filed Aug. 14, 2006, U.S. Provisional Application No. 60/826,061, filed Sep. 18, 2006, and U.S. Provisional Application No. 60/826,068, filed Sep. 18, 2006, which applications are incorporated herein by reference.

### FIELD OF THE INVENTION

The invention relates to the use of air pressure therapy for the treatment and prevention of diseases and conditions that benefit from hypoxic conditioning.

### BACKGROUND OF THE INVENTION

The human vertebral column (spine) comprises a plurality of articulating bony elements (vertebrae) separated by soft tissue intervertebral discs. The intervertebral discs are flexible joints which provide for flexion, extension, and rotation of the vertebrae relative to one another, thus contributing to the stability and mobility of the spine within the axial skeleton.

Intervertebral discs are comprised of a central, inner portion of soft, amorphous mucoid material, the nucleus pulposus, which is peripherally surrounded by an annular ring of layers of tough, fibrous material known as the annulus fibrosus. The nucleus pulposus and the annulus fibrosus together are bounded on their upper and lower ends (i.e., cranially and caudally) by vertebral end plates located at the lower and upper ends of adjacent vertebrae. These end plates, which are composed of a thin layer of hyaline cartilage, are directly connected at their peripheries to the lamellae of the inner portions of the annulus fibrosus. The lamellae of the outer portions of the annulus fibrosus connect directly to the bone at the outer edges of the adjacent vertebrae.

The soft, mucoid nucleus pulposus contains chondrocytes, which produce fibrils of collagen (primarily Type II collagen, but also Types IX and XI) and large molecules of negatively charged, sulfated proteoglycans. The collagenous components of the nucleus pulposus extracellular matrix comprise a scaffold that provides for normal cell (i.e., chondrocyte) attachment and cell proliferation. The term matrix as used herein refers to a composition which provides structural support for, and which facilitates respiration and movement of nutrients and water to and from, an intervertebral disc. The negatively charged proteoglycan component of the nucleus pulposus extracellular matrix attracts water to form a hydrated gel, which envelops the collagen fibrils and chondrocyte cells. In the normal healthy nucleus pulposus, water comprises between 80-90% of the total weight.

The nucleus pulposus thus plays a central role in maintaining normal disc hydrodynamic function. The large molecular weight proteoglycans are contained within the nucleus pulposus by the annulus fibrosus and by the vertebral end plates, and they attract water into the nucleus through sieve-like pores in the end plates. The resulting osmotic pressure within each disc tends to expand it axially (i.e., vertically), driving the adjacent vertebrae further apart. On the other hand, mechanical movements resulting in axial compression, flexion, and rotation of the vertebrae exert forces on the intervertebral discs, which tends to drive water out of the nucleus pulposus. Water movements into and out of an intervertebral disc under the combined influence of osmotic gradients and mechanical forces constitute hydrodynamic functions important for maintaining disc health. Such movement of fluids associated with intervertebral discs is well-documented under the diurnal effect in humans. The discs lose fluids due to compression from the weight of the body when upright during the day and regain fluid as the discs are free from pressure while the body is horizontal or prone during sleep.

Movement of solutes in the water passing between discs and vertebrae during normal hydrodynamic function facilitates chondrocyte respiration and nutrition within the discs. This function is critical to chondrocyte survival since nucleus pulposus tissues of intervertebral discs are avascular (the largest such avascular structures in the human body). Maintaining sufficient water content in the nucleus pulposus is also important for absorbing high mechanical (shock) loads, for resisting herniation of nucleus pulposus matter under such loads, and for hydrating the annulus fibrosus to maintain the flexibility and strength needed for spine stability.

Facilitating the movement of fluids to the discs, the vertebral plates on either side of each disc support the majority of the disc's nutrition via the capillary beds located on the cartilaginous endplate. The capillary beds receive blood flow from the arteries supplying the vertebrae. Neovascularity has been associated with injured discs, however healthy discs isolated from cadavers also show vascularization via the capillary beds. [Martin M D, Boxell C M, and Malone D G, Pathophysiology of Lumbar Disc Degeneration: A Review of the Literature, Neruosurg. Focus 13(2):E1, 2002]. Additional studies have shown that a reduction in the size and density of the capillary beds due to occlusion from a variety of pathologies contributes to nutrient and fluid deprivation in the discs and subsequent degenerative disc disease. [Benneker L M, Heini P F, Alini M, Anderson S E, and Ito K, 2004 Young Investigator Award Winner: vertebral endplate marrow contact channel occlusions and intervertebral disc degeneration, Spine 30(2):167-73 (2005); Urban J P, Smith S, Fairbank J C, Nutrition of the intervetebral disc, Spine 29(23):2700-9 (2004)].

Normal hydrodynamic functions are compromised in degenerative disc disease (DDD). DDD involves deterioration in the structure and function of one or more intervertebral discs and is commonly associated with aging and spinal trauma. Although the etiology of DDD is not well understood, one consistent alteration seen in degenerative discs is an overall decrease in proteoglycan content within the nucleus pulposus and the annulus fibrosus. The loss in proteoglycan content results in a concomitant loss of disc water content. Reduced hydration of disc structures weakens the annulus fibrosus, predisposing the disc to intervertebral trauma such as herniation. Herniation frequently results in extruded nucleus pulposus material impinging on the spinal cord or nerves, causing pain, weakness, and in some cases, permanent disability. Spinal cord injuries are characterized by damage to the neural tissue of the spinal cord. Such injuries may result from an impact injury, associated auto-immune or cancerous conditions (i.e.: tumors, etc.), and/or the result of manipulation associated with certain surgical procedures. Depending upon the site of the injury, the impaired function of the associated neurons can result in a decrease in muscular response or function, and more severe damage can result in partial or complete paralysis. Injuries located near the top of the spinal cord (the cervical region) often lead to paralysis and typically include impaired breathing due to loss of diaphragm function. Injuries located in the central cord (thoracic region) and lower (lumbar region) result in a variety of impairments which tend to correspond to the sections of the body proximal to the injury site and lower.

As with spinal injuries, local inflammation and swelling often result from localized injury, trauma, or infection and the same events can also be the cause of systemic inflammation. Inflammation is often characterized by increased redness, swelling, temperature, pain, and some loss of function in the affected area. Breakdown or dysfunction in the regulation of inflammatory responses can also lead to chronic diseases such as arthritis, inflammatory bowel diseases, asthma, allergic responses, and a host of other inflammatory conditions.

Wound healing represents another significant health issue and entails a complex biological process regardless of causation. In general, the wound is cleaned by infiltrating cells and fluids during the associated inflammatory response. This initial inflammatory phase is followed by a proliferative phase where different cell types provide the necessary factors and tissue environment for wound healing or filling-in by appropriate cells such as fibroblasts, keratinocytes, and a variety of others. Additional events such as angiogenesis and contraction of the wound as epithelial cells gradually fill-in the wound also occur. This phase tends to last about 7-10 days depending upon the severity of the wound and the efficiency of the inflammatory phase. Circumstances such as older age, immunodeficiency, as well as stress, and other environmental factors can affect wound healing. Extended exposure of the wound leads to increased possibilities of infection, adverse inflammatory effects, as well as scarring and possibly chronic wounds. Generally, the wound healing process resolves with the maturation and remodeling phase. Collagen is replaced, remodeled, and cross-linked, thereby increasing the strength of the newly developed tissue and unnecessary blood vessels, cells and tissues are slowly removed from the wound site. This final phase can last up to several years as the body tends to the final healing stage.

Treatments for wounds typically involve the application of antibiotics as well as agents which provide protection from the external environment such as bandages, stitches, second skin, sealants, or other creams and salves. Additionally, numerous compounds are also available for treatment of inflammation in the early phase of wound healing, often in combination with steroidal anti-inflammatory compounds or pharmaceuticals.

### SUMMARY OF THE INVENTION

The present invention provides for a method of administering pressure changes to a user for the treatment of spinal cord injury, the treatment of intervertebral discs, the treatment of inflammation and swelling, and the treatment of wounds. Treatment as used herein includes application of the disclosed methodologies for prevention, prophylacetic treatment, current treatment, amelioration, and recovery. Application of the disclosed methodologies aids in recovery from acute spinal cord trauma and associated surgery. Further, application of disclosed methodologies strengthens intact neuronal pathways and improves associated neuronal and muscle function and control as well as intervertebral disc hydration and health. Similarly, reduction in inflammation and would healing are improved by application of the disclosed methodologies.

One aspect of the invention is the administration of one or more Cyclic Variations in Altitude Conditioning Sessions (CVAC Session(s)) for the treatment of spinal cord injury. In an embodiment of the invention, at least one CVAC session is administered prior to the occurrence of a spinal cord injury, in anticipation of spinal cord surgery, or in anticipation of any surgery that may impact the spinal cord in any way. CVAC sessions may be administered in defined intervals or at random occurrences. In additional embodiments, CVAC sessions are administered following a spinal cord injury. The effect of such administration is a lessening of spinal cord injury symptoms, reduction in continued damage to neuronal and spinal cord tissues, and/or reducing the detrimental effects of spinal cord injuries.

Another aspect of the invention is the administration of one or more CVAC Sessions for the hydration of intervertebral discs. In an embodiment of the invention, at least one CVAC session is administered prior to the occurrence of an intervertebral disc trauma, in anticipation of spinal cord surgery, or in anticipation of any surgery that may impact the spinal cord or intervertebral discs in any way. CVAC sessions may be administered in defined intervals or at random occurrences. In additional embodiments, CVAC sessions are administered following an intervertebral disc trauma, surgery, or associated spinal surgery. The effect of such administration is the modulation of intervertebral disc hydration, reduction in continued damage to intervertebral discs and spinal cord tissues, and/or reducing the detrimental effects of intervertebral disc trauma.

Yet another aspect of the invention is the administration of CVAC sessions for the treatment of inflammation or swelling or combinations thereof. In an embodiment of the invention, at least one CVAC session is administered prior to the occurrence of inflammation or swelling or in anticipation of surgery, or combinations thereof. CVAC sessions may be administered in defined intervals or at random occurrences. In additional embodiments, CVAC sessions are administered following inflammation or swelling caused by injury, trauma, infection, and/or the breakdown or dysfunction of the immune system. The effect of such administration is a lessening of inflammation symptoms, a lessening of swelling, reducing the continued damage to inflamed or swollen tissues, or reducing the detrimental effects of inflammation or swelling, and combinations thereof.

An additional aspect of the invention is the administration of CVAC sessions for the treatment of wounds. In an embodiment of the invention, at least one CVAC session is administered to improve or reduce the actual healing time of a wound. CVAC sessions may be administered in defined intervals or at random occurrences. The effect of such administration is a lessening of healing time for a wound as well as improvement of wound healing.

Further embodiments of the invention include the reduction of healing time of a wound, the increased drainage of fluids or toxins of combinations thereof from the affected areas, and/or the modulation of genetic elements and resultant expression of molecules involved in inflammatory and immune responses.

A CVAC session consists of a set of targets which are pressures found in the natural atmosphere. A CVAC session includes start and end points and more than one target which is executed between the start and end points, These targets are delivered in a precise order, and are executed in a variety of patterns including, but not limited to, cyclic, repeating, and/or linear variations. The starting points and ending points in any CVAC session are preferably the ambient pressure at the delivery site. The targets inherent in any CVAC session are connected or joined together by defined transitions. These transitions are either rises in pressure or falls in pressure, or a combination of the two. Additional targets which modulate time, temperature, or humidity are also run concurrently, sequentially, or at other intervals with the pressure targets when such additional targets and conditions are desired.

In an additional embodiment, including the aforementioned embodiments and aspects, the targets of the CVAC sessions include pressure, temperature, time, and humidity parameters. Parameters of targets and sessions can be customized to individual needs. In yet another embodiment of the invention, including the aforementioned embodiments and aspects, CVAC sessions are administered in combination with pharmaceutical regimens for the treatment, prevention or amelioration of spinal cord injury. Further embodiments, including the aforementioned embodiments and aspects, include administration of CVAC sessions in combination with alternative therapies and non-pharmaceutical therapies for the treatment spinal cord injuries, intervertebral discs, inflammation, and wound healing.

### BRIEF DESCRIPTION OF THE DRAWINGS

FIG. 1A depicts a graphed profile of the various pressures applied over time during an exemplary CVAC session. The Y-axis represents atmospheric pressure levels and the X-axis represents time. The varying pressures, as indicated by the changes in values on the Y-axis, were applied for various lengths of time, as indicated by changes values on the X-axis. The exemplary CVAC session depicted in FIG. 1A was 20 minutes in length.

FIG. 1B depicts a different graphed profile of the pressures applied over time during another exemplary CVAC session. The Y-axis again represents atmospheric pressure levels and the X-axis represents time. Different pressures were again applied, as indicated by changes in value on the Y-axis, for various lengths of time, as indicated by the changes in values on the X-axis. This exemplary CVAC session was also 20 minutes in length.

### DETAILED DESCRIPTION OF THE INVENTION

While oxygen deprivation of the body or specific tissues can cause tissue damage, and even death, controlled deprivation of oxygen to the body and/or specific tissues has been shown to be beneficial when imposed for specific periods of time under particular conditions. In practice, most current hypoxic conditioning protocols utilize static pressures for blocks of time ranging from 30 minutes to an hour or more to achieve the desired and reported responses. Hypoxic conditioning may be provided by decreased oxygen levels in the atmosphere or by a reduction in atmospheric pressure (hypobaric conditions), thus reducing the availability of oxygen for efficient respiration. Both methods can provide beneficial results including protection of tissues from damage due to injury and ischemia.

Moderate static hypoxic preconditioning is known to provide protection from ischemic damage via tolerance. When the environmental oxygen levels are reduced (hypoxia), downstream effects include protection from damage due to subsequent hypoxia. [Sharp, F., et al., Hypoxic Preconditioning Protects against Ischemic Brain Injury, NeuroRx: J. Am. Soc. Exp. Neuro., Vol. 1: 26-25 (2004)]. This tolerance is not yet completely understood, but it has been linked to various cellular mechanisms and molecules, including, but not limited to, molecules such as erythropoietin (EPO), hypoxia-inducible factor (HIF), Tumor Necrosis Factor (TNF), glycogen, lactate, and others. [Sharp, F., et al., Hypoxic Preconditioning Protects against Ischemic Brain Injury, NeuroRx: J. Am. Soc. Exp. Neuro., Vol. 1: 26-25 (2004)]. Additionally, beneficial static hypoxic conditioning is not purely additive. Administration of sequential sessions can have detrimental effects. Oxygen concentrations that are too low result in detrimental effects to the tissues as well as the entire body. Similarly, hypoxic conditioning of longer durations can have detrimental effects in addition to providing some desired beneficial effects [Sharp, F., et al., Hypoxic Preconditioning Protects against Ischemic Brain Injury, NeuroRx: J. Am. Soc. Exp. Neuro., Vol. 1: 26-25 (2004)].

Initial understanding in the art about the effects of hypoxia focused on increased oxygenation of the blood via increased production of red blood cells mediated by increases in EPO production. While increases in EPO production are believed to increase red blood cell production, its effects are not limited to this activity. Additional studies also show protective activity for EPO in white and gray matter (brain and spinal cord tissue), inflammatory and demyelinating conditions, and other various ischemic events. [Eid, T. and Brines, M., Recombinant human erythropoietin for neuroprotection: what is the evidence?, Clin. Breast Cancer, 3 Suppl. 3:S109-15, December 2002]. Furthermore, molecules such as HIF, induced by hypoxia, regulate EPO production in addition to a variety of other activities including metabolism, angiogenesis, and vascular tone—the stimulation of which may all play a role in protecting tissue from subsequent hypoxic damage both prophylactically and post-ischemic or traumatic events. [Eckardt K. U., Kurtz, A., Regulation of erythropoietin production, Eur. J. Clin. Invest., 35(Supp. 3):13-19, (2005)].

Vascular endothelial growth factor (VEGF) is a known hypoxia induced protein under the control of HIF-1α VEGF has been shown to have direct neuroprotective effects on mammalian spinal cord neurons following spinal cord injury. [Ding X M, et al., Neuroprotective effect of exogenous vascular endothelial growth factor on rat spinal cord neurons in vitro hypoxia, Chin. Med. J (Engl), 118(19):1644-50, Oct. 5, 2005]. Intermittently administered static hypoxic conditions have been shown to augment phrenic motor activity (the phrenic nerve controls breathing via the diaphragm among other organ functions) and exerted such effects as far out as 8 weeks after spinal cord injury. [Golder, F J and Mitchell, G S, Spinal synaptic enhancement with acute intermittent hypoxia improves respiratory function after chronic cervical spinal cord injury, J. Neurosci., 25(11):2925-32, Mar. 16, 2005]. Amelioration of spinal cord damage can enhance respiratory motor output and stimulated neural plasticity within the damaged spinal cord, however extended hypoxia can result in detrimental effects. Thus, chronic, intermittent static hypoxic conditions produce the most beneficial results. [Fuller, D., et al., Synaptic Pathways to Phrenic Motoneurons Are Enhanced by Chronic Intermittent Hypoxia after Cervical Spinal Cord Injury, J. Neurosci., 23(7):2993-3000, Apr. 1, 2003]. Additional studies have shown increased expression and resultant levels of glycolytic enzymes and VEGF following static hypoxic interval treatments administered post-spinal cord injury. The effect is to induce hypoxic tolerance and vascularity of the injured spinal cord. [Xiaowei H, et al., The experimental study of hypoxia-inducible factor-1 alpha and its target genes in spinal cord injury, Spinal Cord, 44(1):35-43, January 2006].

Current treatments for acute spinal cord injury encompass primarily pharmaceutical therapies, physical therapy and surgical intervention. Surgical intervention is quite traumatic to the body and can result in additional medical complications, especially where the body is already severely weakened or compromised due to the severity spinal cord injury and/or the over-all health and condition of the patient. Pharmaceuticals such as corticosteroids may also be used to treat acute spinal cord injuries, but as with surgery, pharmaceuticals can bring on additional concerns due to negative side-effects from the compound itself, length of treatment, and unforeseen, individual reactions to the drugs. For example, glucocorticoids administered to relieve inflammation and swelling can exacerbate the excitotoxic phase of neural injury in addition to the known detrimental effects of extended use, thus limiting their effectiveness in limiting the initial damage and their potential for long-term therapy. Physical therapy can also ameliorate some of the damaging effects of spinal cord injury, however this treatment primarily addresses the affected muscle groups rather than the spinal cord itself and amelioration of neuronal damage. Notably, a majority of spinal injuries are also incomplete, thus the damage has not severed the spinal cord completely and some intact neuronal pathways remain. Currently, physical therapy and most pharmaceutical regimens are unable to adequately address the need to strengthen these remaining pathways for improved neurological function and control.

Current treatments for disc degeneration encompass primarily pharmaceutical therapies, physical therapy and surgical intervention. As above, surgical intervention is quite traumatic to the body Pharmaceuticals such as corticosteroids may also be used to treat disc degeneration, but as with surgery, pharmaceuticals can bring on additional concerns due to negative side-effects from the compound itself, length of treatment, and unforeseen, individual reactions to the drugs. For example, glucocorticoids administered to relieve inflammation and swelling can exacerbate the excitotoxic phase of neural injury in addition to the known detrimental effects of extended use, thus limiting their effectiveness in limiting the initial damage and their potential for long-term therapy. Physical therapy can also ameliorate some of the damaging effects of disc degeneration; however this treatment primarily addresses the affected muscle groups rather than the spinal cord, amelioration of disc damage, and vertebral plate damage.

Treatments for inflammation and swelling similarly utilize pharmaceuticals and typically involve the administration of steroids in a variety of formulations and methods. Additionally, numerous non-steroidal compounds are also available for treatment of inflammation, often in combination with steroidal anti-inflammatory compounds or pharmaceuticals. As with inflammation, current treatments for wounds encompass primarily anti-inflammatory therapies, antibiotics, and physical protections or interventions (bandages, sealants, stitches, etc.). Pharmaceuticals such as corticosteroids and other steroid-based anti-inflammatories can bring on additional concerns due to negative side-effects from the compound itself, length of treatment, and unforeseen, individual reactions to the drugs. For example, glucocorticoids, administered to relieve inflammation and swelling, have known detrimental effects associated with extended use thus limiting their effectiveness and their potential for long-term therapy, and inhibition of the inflammatory response is not always beneficial to wound healing.

Alternative therapies such as oxygen deprivation are known to provide some beneficial effect as well. While oxygen deprivation of the body or specific tissues can cause tissue damage, and even death, controlled deprivation of oxygen to the body or specific tissues or a combination thereof has been shown to be beneficial when imposed for specific periods of time under particular conditions. Hypoxic conditioning may be provided by decreased oxygen levels in the atmosphere or by a reduction in atmospheric pressure (hypobaric conditions), thus reducing the availability of oxygen for efficient respiration. Both methods can provide beneficial results including prevention of damage due to inflammation and swelling. However, all current forms of hypoxic conditioning involve applications of static pressures and involve relatively long periods of application.

There is a need for alternative therapies for spinal cord injuries, intervertebral disc treatments, inflammation, and wound healing. Further there is a need for such therapies without the potential negative side-effects of pharmaceutical regimens. Alternatively, there is a need for such therapies that could lessen the negative side-effects of pharmaceutical regimens by altering pharmaceutical regimens, could work beneficially with pharmaceutical regimens, or could work synergistically when used in combination with pharmaceutical regimens. There is a need for hypobaric or hypoxic conditioning which maximizes the beneficial effects within short treatment periods that do not lead to the detrimental effects of such conditioning as found with current methods of static hypobaric conditioning. There is a further need for such hypobaric or hypoxic conditioning that utilizes multiple and/or varying pressures throughout the conditioning. There is yet a further need for hypobaric or hypoxic conditioning that incorporates vaso-pneumatic considerations in addition to the hypoxic considerations. The inventions disclosed herein provide for such needs and are unique and superior to all previous forms of hypobaric conditioning. Among the many benefits, the application of CVAC sessions provides beneficial effects of hypobaric conditioning in a greatly reduced time frame due to the unique combination of pressures and time. Additionally, CVAC sessions provide for vaso-pneumatic beneficial effects in the same time frame.

A Pressure Vessel Unit (PVU) is a system for facilitating pressure changes accurately and quickly in the environment surrounding a user. A PVU can provide both reduced and increased atmospheric pressures. An example of a unique PVU and associated methods for controlling the pressure within such a PVU are described in U.S. Patent Publication No. 2005/0056279 A1 and incorporated herein by reference. A variety of PVUs may be used in conjunction with the methods disclosed herein, including but not limited to those described in the U.S. Patent Publication No. 2005/0056279, such as variable or fixed pressure and temperature hypobaric units. Other pressure units or chambers will be known to those of skill in the art and can be adapted for use with the disclosed methodologies.

### Methodology of the Cyclic Variations in Altitude Conditioning (CVAC) Program

The methodology of the present invention encompasses a set of pressure targets with defined transitions. Additional targets can be included such as temperature or humidity, and these targets can be implemented concurrently, prior to, or subsequent to the pressure targets. The permutations of targets are customizable to the individual and condition to be treated. Some of the terms relating to this methodology are defined below for a better understanding of the methodology as used in the context of the present invention.

A CVAC Program: Every user will respond in a unique manner to changes in air pressure, temperature and oxygen levels that occur during cyclic variations in altitude conditioning. This necessitates a customized approach to delivering a highly effective and efficacious CVAC program to each user The program consists of a set of sessions, which are administered to the user as a serial round or cycle. This means that a user may have a session that they start and repeat a given number of times and then proceed to the next scheduled session which will be repeated a given number of times. A program may contain a set of one or more sessions, each of which preferably has a repetition schedule. The sessions are preferably delivered in a scheduled order, which repeats itself like a loop such that the user is administered one session at a time for a specified number of times. The user is then administered the next scheduled session a specified number of times. This process is preferably repeated until the user is administered the last element of the scheduled sessions set. When the requisite repetitions have been accomplished, preferably the process repeats itself beginning at the first element of the scheduled sessions set. A session or groups of sessions may be repeated multiple times before changing to a subsequent session or group of sessions, however, sessions may also be administered as few as one time before beginning the next session in the sequence. Subsequent sessions can contain targets that are identical to the previous session, or they can implement new permutations of desired targets. The combination of sessions and targets within sessions is customizable based on the desired physiological outcome and assessment of the user. Alternatively, a user may also modulate the parameters of a CVAC session, in certain embodiments from within the unit, thus providing for real-time user feedback and alterations. As used in reference to parameter of a CVAC session, modulation includes any changes, positive and negative, made to the parameters of the CVAC session. The parameters are described herein. This comprises a Cyclic Variations in Altitude Conditioning (CVAC) Program.

A CVAC Session: A CVAC Session comprises of a set of targets which are multiple atmospheric pressures, and a CVAC session includes start and end points, and more than one target which is executed between the start and end points. These targets are delivered in a precise order that may vary and are executed in a variety of patterns including, but not limited to, cyclic, repeating, and/or linear variations. When a target is executed as contemplated herein, executed includes a change in pressure from one pressure value to another pressure value within a CVAC device as also described herein. The methodologies described herein are superior to previously described static hypobaric pressure therapies in multiple ways, which can include reduced time frames of application and unique variations and combinations of atmospheric pressures. Furthermore, CVAC sessions can also provide beneficial effects via the vas-pneumatic properties associated with the application of such sessions. The starting points and ending points in any CVAC Session are preferably the ambient pressure at the delivery site. The targets inherent in any CVAC Session are connected or joined together by defined transitions. These transitions are either increases in pressure (descent) or decreases in pressure (ascent), or a combination of the two. The nature of any transition may be characterized by the function of “delta P/T” (change in pressure over time). Transitions may be linear or produce a waveform. Preferably, all transitions produce a waveform. The most desirable waveforms are Sine, Trapezoidal and Square. Additional targets which modulate time, temperature, and/or humidity are also run concurrently, sequentially, or at other intervals with the pressure targets when such additional targets and conditions are desired. The entire collection of targets and transitions are preferably delivered in a twenty minute CVAC Session, although the time of each session may vary in accordance with the desired outcome of the administration of the CVAC Sessions. For example, CVAC sessions may be administered over minute increments such as 5, 10, 15, 16, 17, 18, 19, 20, 25, 30 minutes and/or more. The length of each CVAC Session is customizable for each user.

A Set-Up Session: The Set-Up Session may also be considered a Program. It is a single Session designed to prepare a new user for the more aggressive maneuvers or transitions encountered in the subsequent Sessions that the user will undergo. The Set-Up session accounts for all ages and sizes and conditions, and assumes a minimal gradient per step exercise that allows the ear structures to be more pliant and to allow for more comfortable equalization of pressure in the ear structures. The purpose of the Set-Up session is to prepare a new user for their custom Program based upon the group into which they have been placed. The function of the Set-Up session is to qualify a user as being capable of adapting to multiple pressure changes in a given Session with acceptable or no discomfort. Set-Up session transitions may be linear or produce a waveform. Preferably, all transitions are linear. This is accomplished by instituting a gradient scale increase in pressure targets from very slight to larger increments with slow transitions increasing until a maximum transition from the widest difference in pressure targets is accomplished with no discomfort. The structure of a preferred Set-Up session is as follows: as with any Session, the starting point and ending point is preferably at ambient pressure. A target equivalent to 1000 ft above ambient is accomplished via a smooth linear transmit. A second target equivalent to 500 ft less than the first target is accomplished via a slow to moderate transmit. These two steps are repeated until the user returns a “continue” or “pass” reply via an on-board interface. When the user has indicated that they are prepared to continue, the initial target (1000 ft) is increased by a factor of 500 ft, making it 1500 ft. The secondary target (500 ft less than the first target) remains the same throughout the session until the exit stage is reached. Each time the user indicates that they are ready to increase their gradient, the target is increased by a factor of 500 ft. At this time, the transmits remain the same but the option of increasing gradient (shorter time factor) in the transmits is available. A user preferably has the option of resuming a lower gradient if desired. There can be an appropriate icon or pad that allows for this option on the on-board interface display screen. Preferably, the Set-Up Session lasts no longer than 20 minutes. A Set-Up session typically runs for twenty minutes maximum and executes a final descent to ambient atmospheric pressure upon beginning the last transmit. The Set-Up session is a new user's Program until the user is able to fully complete the Set-Up session (that is to continue the targets and transmits to the highest gradient) with no interrupts or aborts. When administering CVAC sessions for medical treatment, Set-Up sessions may be customized to suit the requirements of their medical condition. The determination of the appropriate Set-Up Session can be made with guidance from or consultation with a user's qualified health professional, such as a treating physician.

The Interrupt: During any phase in a Session wherein a user desires to stop the Session at that point for a short time, they may do so by activating an icon or other appropriate device on the on-board interface touch screen or control pad. This will hold the Session at the stage of interruption for a predetermined time period, such as a minute, at which time the Session will continue automatically. Preferably, a Session may be interrupted three times after which a staged descent will occur and the user will be required to exit the pressure vessel. The user's file will be flagged and the user will be placed back on the Set-Up Sessions until they can satisfactorily complete it. A warning or reminder may be displayed on the screen each time an interrupt is used that informs the user of how many times interrupt has been used and the consequences of further use. During any session, be it a Set-Up session or other type of session, a staged descent is also available if the user develops ear or sinus discomfort or wishes to terminate the session for any reason. A staged descent is characterized by slow, 1000 ft sine wave descent transmits with re-ascensions of 500 ft at each step. The descents can be of greater or lesser transmits but the ratio is usually about 1.5:1. At any time during the staged descent, the user can interrupt the descent and hold a given level or resume a previous level until comfort is achieved. The user may also re-ascend at their option if the staged descent is too aggressive. Any re-ascension is done in stages as described above. The user can subsequently indicate a “continue” on the descent and the staging will resume. This stepping continues until ambient pressure is reached whereupon the canopy opens such that the user can exit the pressure vessel.

The Abort: When a user wishes to end a session immediately and quickly exit the pressure vessel, the abort function can be activated. Touching the “abort” icon on the on-board interface touch pad/screen enables this option. A secondary prompt is activated acknowledging the command and asking the user if they are sure they want to abort. The user indicates their commitment to the command by pressing “continue” or “yes”. The program is aborted and a linear moderate descent is accomplished to ambient pressure whereupon the canopy opens and the user exits. The user's file is flagged. The next time the user comes in for their session, the user is asked whether the abort was caused by discomfort. If yes, the user is placed back on the Set-Up session program. If no, the user is asked if they wish to resume their regularly scheduled session. The client is given the option of resuming their regularly scheduled Session or returning to the Set-Up session.

### Program and Target Criteria, Including Medically Significant Criteria

Preferably, a user is categorized into a group of users having similar body-types with similar characteristics based upon answers to a questionnaire. The information from the questionnaire guides the construction of custom CVAC programs for each individual. When administering CVAC programs for spinal cord injury therapy, the medical status of the user can also be used to determine appropriate pressures and additional parameters (such as duration, temperature, or humidity) of the targets. Custom session targets may be administered based upon the medical condition and therapy desired. The acceptable and appropriate target parameters may be obtained as described herein and through consultation with the user's physician or other appropriate health-care provider prior to designing session targets and administering a CVAC session. However the known contraindications of CVAC are similar to those of commercial air travel, allowing for a broad range of application.

Methods of Treatment:

In one aspect of the invention, CVAC sessions for the treatment of spinal cord injury are administered preferably for at least 10 minutes, and more preferably at least 20 minutes, with variable frequency. Additional administration periods may include, but are not limited to, about 10 minutes, about 20 minutes, about 30 minutes, about 40 minutes, about 60 minutes, between 10 and 20 minutes, between 20 and 30 minutes, between 30 and 60 minutes, and between 60 and 120 minutes. Frequencies of sessions or series of sessions may include, but are not limited to, daily, monthly, or when medically indicated or prescribed. The frequency and duration of the sessions can be altered to suit the medical condition to be treated, and CVAC sessions may be administered as single sessions, or as a series of sessions, preferably with a Set-Up Session as described herein. For example, the frequency of sessions or series of sessions can be administered 3 times a week for 8 weeks, 4 times a week for 8 weeks, 5 times a week for 8 weeks, or 6 times a week for 8 weeks. Additional frequencies can be easily created for each individual user. Similarly, the targets in the sessions can also be altered or adjusted to suit the individual and medical condition to be treated. If at any time the user or attendant determines that the session is not being tolerated well, an abort may be initiated and the user brought down safely and exited. The permutations of targets can be customized to the individual, and may again be identified with the help of any person skilled in the art, such as a treating physician. Furthermore, the variations may be administered in regular intervals and sequence, as described, or in random intervals and sequence. The variations in number, frequency, and duration of targets and sessions can be applied to all methods of treatment with CVAC described herein.

In an embodiment of the present invention, Cyclic Variations in Altitude Conditioning Program is used to prophylactically treat users who are anticipating spinal cord surgery or any surgery that may impact the spinal cord. In anticipation of spinal cord surgery, CVAC is administered to increase the oxygenation of the spinal cord, increase the production of HIF's, and stimulate other associated physiological processes affected by CVAC treatment such as fluid movement and reduction in swelling. Treatment is administered through the use of one or more CVAC sessions. Such sessions may be user defined or custom-defined with input from the user's physician. CVAC sessions may be administered in advance of any such surgeries or treatments to help reduce or prevent any damaging effects.

In another aspect of the present invention, CVAC sessions are administered for the treatment of intervertebral discs. As described herein, treatment of intervertebral discs includes, but is not limited to, the hydration of intervertebral discs as well as the prevention, treatment or amelioration of intervertebral disc trauma. Similarly, the treatment of intervertebral discs includes prophylacetic administration as well as administration for treatment and maintenance. CVAC sessions for the treatment of intervertebral discs are administered preferably for at least 10 minutes, and more preferably at least 20 minutes, with variable frequency. Additional administration periods may include, but are not limited to, about 10 minutes, about 20 minutes, about 30 minutes, about 40 minutes, about 60 minutes, between 10 and 20 minutes, between 20 and 30 minutes, between 30 and 60 minutes, and between 60 and 120 minutes. Frequencies of sessions or series of sessions may include, but are not limited to, daily, monthly, or when medically indicated or prescribed. The frequency and duration of the sessions can be altered to suit the medical condition to be treated, and CVAC sessions may be administered as single sessions, or as a series of sessions, preferably with a Set-Up Session as described herein. For example, the frequency of sessions or series of sessions can be administered 3 times a week for 8 weeks, 4 times a week for 8 weeks, 5 times a week for 8 weeks, or 6 times a week for 8 weeks. Additional frequencies can be easily created for each individual user. Similarly, the targets in the sessions can also be altered or adjusted to suit the individual and medical condition to be treated. If at any time the user or attendant determines that the session is not being tolerated well, an abort may be initiated and the user brought down safely and exited. The permutations of targets can be customized to the individual, and may again be identified with the help of any person skilled in the art, such as a treating physician. Furthermore, the variations may be administered in regular intervals and sequence, as described, or in random intervals and sequence. The variations in number, frequency, and duration of targets and sessions can be applied to all methods of treatment with CVAC described herein.

In another embodiment of the present invention, Cyclic Variations in Altitude Conditioning Program is used to prophylactically treat users who are anticipating intervertebral disc surgery or any surgery that may impact the spinal cord and/or the intervertebral discs. In anticipation of such surgery, CVAC is administered to increase the oxygenation of the vertebral endplates, increase the production of HIF's, and stimulate other associated physiological processes affected by CVAC treatment such as fluid movement and reduction in swelling. Such movement of fluids further facilitates the hydration of the intervertebral discs. Treatment is administered through the use of one or more CVAC sessions. Such sessions may be user defined or custom-defined with input from the user's physician. CVAC sessions may be administered in advance of any such surgeries or treatments to help reduce or prevent any damaging effects.

In yet another aspect of the present invention, Cyclic Variations in Altitude Conditioning Program is used to treat users who are experiencing any form of inflammation or swelling and combinations thereof, including in anticipation of such conditions. Thus, treatment of inflammation includes administration of at least one CVAC session prior to inflammation or swelling and following the onset of inflammation or swelling, irrespective of the cause. In one embodiment, CVAC is administered to increase the oxygenation of the inflamed or swollen tissue, increase the production of HIF's, and stimulate other associated physiological processes affected by CVAC treatment such as fluid (lymph, blood, or other bodily fluids) movement and reduction in swelling. Treatment is administered through the use of one or more CVAC sessions. Such sessions may be user defined or custom-defined with input from the user's physician. In another embodiment, CVAC sessions may be administered in advance of, or following any surgeries or other treatment regimens to help reduce or prevent any damaging effects relating to inflammation and swelling.

A further aspect of the invention is the administration of CVAC sessions for wound healing. In one embodiment of the present invention, Cyclic Variations in Altitude Conditioning Program is used to treat users who have wounds of any type, including but not limited to wounds such as surface wounds, cuts, scratches, lacerations, burns, ulcerations, punctures, stabbings, and projectile wounds such as those from gun-shots or other firearms. CVAC is administered to increase the oxygenation of the wounded tissue, increase the production of HIF's, and/or stimulate other associated physiological processes affected by CVAC treatment such as fluid (lymph, blood, or other bodily fluids) movement and reduction in swelling. Further, CVAC sessions are used to exert micromechanical force on wounded tissues to stimulate cell proliferation. Use of CVAC sessions for treatment of wound healing includes use of such sessions prior to a wound, following the infliction of a wound, use wherein the length of the inflammatory phase of wound healing is reduced, use wherein the length of the proliferative phase of wound healing is reduced, and use wherein the length of the maturation and remodeling phase of wound healing is reduced.

Treatment is administered through the use of one or more CVAC sessions. Such sessions may be user defined or custom-defined with input from the user's physician. CVAC sessions may be administered in advance of any surgeries or other treatment regimens to help reduce or prevent any damaging effects. CVAC sessions may also be used in combination with pharmaceutical regimens or non-pharmaceutical therapies such as surgery, bandages, sealants, or topical creams, salves, etc. and combinations thereof to aid in or improve wound healing.

Although not limited, CVAC sessions are believed to act like a vaso-pneumatic pump on the user's body, thus stimulating flow of fluids in the body, including but not limited to blood and lymphatic fluids. The negative and positive pressures imposed by the CVAC session affect the fluid flow or movement within a body, thus improving the delivery of beneficial nutrients, immune factors, blood, and oxygen while also improving the removal of harmful toxins, fluids, and damaged cells or tissues. Additionally, CVAC sessions are believed to provide increased blood flow, increased red blood cell counts, angiogenic and protective cellular responses, EPO production, and HIF production can aid in recovery and repair of damaged tissues. The combination of the beneficial effects of CVAC sessions results in treatment and improved recovery from inflammation and swelling, and similarly benefits all the aforementioned aspects and embodiments.

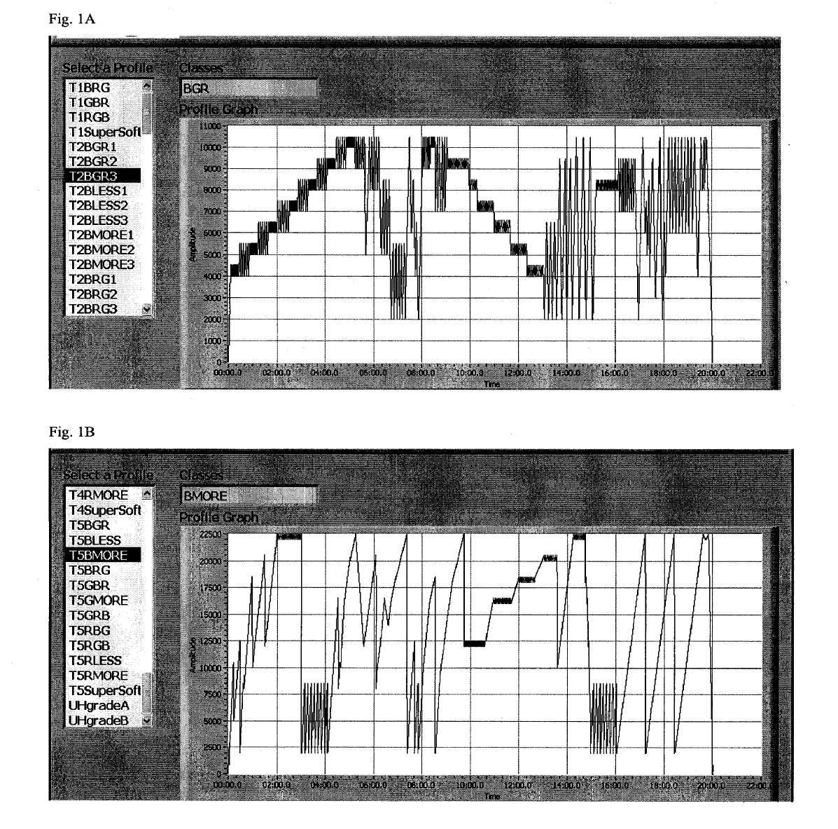

Specific examples of a CVAC session are shown graphically in FIGS. 1A and 1B. In both figures, the parameters of the program are shown as a line graph with axes that correspond to time α-axis) and pressure change (y-axis).

CVAC sessions for any of the aforementioned aspects and embodiments may also be used in combination with pharmaceutical regimens or non-pharmaceutical therapies such as physical therapy. As described above, CVAC sessions of any combination or permutation can be administered prior to, concurrent with, or subsequent to administration of a pharmaceutical, pharmaceuticals, or non-pharmaceutical therapy. Myriad permutations of pharmaceutical therapies, non-pharmaceutical therapies, and CVAC session combinations are possible, and combinations appropriate for the type of medical condition and specific pharmaceutical may be identified with the help of any person skilled in the art, such as a treating physician.

Efficacy of Treatment

Efficacy of CVAC treatments for the aforementioned aspects and embodiments can be evaluated with a variety of imaging and assessment techniques known in the art. Examples include methods such as magnetic resonance imaging (MRI) of the affected region, invasive imaging through catheterization, or alternative non-invasive imaging methods. Additional assessment criteria based on physiological markers known in the art include: hematocrit measurement, blood-gas analysis, extent of blood-perfusion of tissues, angiogenesis within tissues, erythropoietin or VEGF production, extent of tissue necropsy, and assessment of motor and/or cognitive abilities following spinal cord injury and treatment. Efficacy of CVAC treatments can also be evaluated with a variety of imaging and assessment techniques known in the art such as magnetic resonance imaging (MRI) of the affected region, invasive imaging through catheterization, or alternative non-invasive imaging methods. By way of example, imaging of the intervertebral discs can identify changes in hydration of said discs in addition to changes in deterioration through visualization of the disc structures.

By example only, when hematocrit is the physiological marker used to assess CVAC efficacy, modulation of hematocrit during or following one or more CVAC sessions is indicative of efficacious CVAC treatment for the treatment, amelioration, or prevention of spinal cord injuries. In one embodiment, an increase in hematocrit is indicative of efficacious CVAC treatment. Conversely, a lack of change in the user's hematocrit (or with any of the physiological markers described herein) does not necessarily indicate that the CVAC treatments are not achieving positive results. Similarly, when blood-gas analysis is the physiological marker used to assess CVAC efficacy, modulation of the dissolved gasses in the blood during or following one or more CVAC sessions is indicative of efficacious CVAC treatment. Typical gasses monitored include oxygen, carbon dioxide, and nitrogen. However, any gas found within the blood may be monitored for assessment of CVAC efficacy. When blood-perfusion of the tissues is the physiological marker used to assess CVAC efficacy, increases in blood volumes and/or blood exchange within tissues during or following one or more CVAC sessions are indicative of the efficacious CVAC treatment. Angiogenesis within affected tissues can also be a physiological marker used to assess CVAC efficacy. Modulation of vessel development within the affected tissues during or following one or more CVAC sessions is indicative of efficacious CVAC treatments. Additionally, initiation or modulation of VEGF expression within affected tissues during or following one or more CVAC sessions is also indicative of efficacious CVAC treatment. Modulation of erythropoietin production following one or more CVAC sessions is also a physiological marker used to assess the efficacy of CVAC treatments. In one embodiment of the present invention, increases in the expression of erythropoietin indicate efficacious CVAC treatments. Still further physiological markers for assessing efficacy of CVAC sessions include modulation of cognitive and/or motor skills during or following one or more CVAC sessions. In one embodiment, improved or increased motor skills are indicative of efficacious CVAC treatment. Similarly, in yet another embodiment improved cognitive skills are indicative of efficacious CVAC treatment.

Extent of tissue necropsy is a further physiological marker used to assess CVAC efficacy. Modulation of tissue necropsy, including repair or efficient removal of affected tissue by known bodily repair systems, pathways, and cascades as well as prevention of initial or continued necrosis, during or following one or more CVAC sessions is indicative of CVAC session efficacy. Still further physical indicators for assessing efficacy of CVAC sessions include modulation of swelling, temperature, or turgidity and combinations thereof during or following one or more CVAC sessions. In one embodiment, reduced swelling, temperature, or turgidity or combinations thereof are indicative of efficacious CVAC treatment. Similarly, in yet another embodiment modulation of immune or inflammation-mediating cells present in the affected tissue, chemokine and cytokine profiles in the affected tissue, or other immune-cell factors or a combination thereof is also indicative of efficacious CVAC treatment. For example, cytokine profiles of interleukins within the affected tissues or body can be monitored to determine efficacy of CVAC treatments. Additional criteria for assessing the efficacy of the aforementioned aspects and embodiments will be known by those of skill in the art and can be employed to assess the beneficial effects of CVAC programs.

Methods for treating spinal cord injury, treating intervertebral discs, treating inflammation and swelling, and treating wounds by administration of various environmental pressure levels for hypoxic conditioning are disclosed herein. Previously described PVU and CVAC methodology is used to implement the methods for treatment of the aforementioned conditions, and alternative PVUs can be used with the disclosed methodologies.

### EXAMPLES

### Example 1

To assess the efficacy of CVAC sessions, four individuals were administered CVAC sessions and their red blood cell counts hematocrit were subsequently measured and the levels recorded. Increases in red blood cell counts are indicative of CVAC session efficacy, and changes in hematocrit similarly indicate changes in erythropoiesis. For the study, CVAC sessions were administered to a group of four individuals for 40 minutes, 4 times a week, over an 8 week period. Red blood cell levels (RBC) were measured at 5 different intervals during the 8 week test period. The results of the study were as follows:
RBC mean increase: 4.7%

The increases in RBC's indicate that CVAC sessions were successful in positively modulating red blood cell counts as well as hematocrit, and both measurements are indicative of increased erythropoiesis. Thus, the administration of CVAC sessions successfully improved erythropoiesis in this 8 week study.

### Example 2

In the same study as example 1, to assess the efficacy of CVAC sessions four individuals were administered CVAC sessions and their hematocrit was subsequently measured and the levels recorded. Changes in hematocrit indicate changes red blood cell concentration as well as indicating changes in erythropoiesis. For the study, CVAC sessions were administered to a group of four individuals for 40 minutes, 4 times a week, over an 8 week period. Hematocrit (HCT) was measured at 5 different intervals during the 8 week test period. The results of the study were as follows:
HCT mean increase: 5.3%

The increases in HCT, both alone in combination with the RBC increase as described in example 1, indicate that CVAC sessions were successful in positively modulating hematocrit levels and are further indicative of increased erythropoiesis. Thus, the administration of CVAC sessions successfully improved erythropoiesis in this 8 week study.

### Example 3

To assess the efficacy of CVAC sessions, 13 individuals, all between the ages of 20 and 40 years old, were administered CVAC sessions and changes in their erythropoietin (EPO) levels were measured. Frequency of CVAC administration was for one hour per day, 5 days per week, for seven weeks. Increases in EPO were measured prior to administration of CVAC and three hours post-administration of CVAC, and EPO concentration is expressed as mIU/ml. Thus changes in EPO can be represented by the formula: deltaEPO=Post-CVAC EPO MIU/ml—pre-CVAC EPO mIU/ml. The study found that EPO levels changed significantly over the study period in the population. Specifically, mean changes in EPO concentration increased from 0.2 mIU/ml following the first 2 weeks of CVAC administration to 2.0 mIU/ml following 8 weeks of the CVAC administration. The significant changes in EPO levels found in the study population indicate that the administration of CVAC sessions can positively modulate EPO production, hence providing an alternative and efficacious method to exogenous EPO administration.

The aspects and embodiments of the present invention described above are only examples and are not limiting in any way. Various changes, modifications or alternations to these embodiments may be made without departing from the spirit of the invention and the scope of the claims.

What is claimed is:

1. A method of treating spinal cord injury in a mammal comprising the step of administering at least one CVAC session, said CVAC session having a start point, an end point and more than one target which is executed between said start point and said end point.

2. The method of claim 1, further comprising the step of measuring efficacy of CVAC sessions via changes in physiological markers.

3. The method of claim 2, wherein the physiological marker measured is selected from among:
hematocrit;
erythropoietin (EPO) production;
blood gas composition;
oxygenation of tissues;
angiogenesis within tissues;
blood-perfusion of tissues;
extent of tissue necropsy following a spinal cord injury;
motor function;
or neuron function.

4. The method of claim 1, further comprising the step of measuring efficacy of the CVAC sessions by non-invasive imaging.

5. The method of claim 1, further comprising the step of measuring efficacy of the CVAC sessions by invasive imaging.

6. The method of claim 1 wherein the user can modulate the parameters of a session.

7. A method of treating intervertebral discs in a mammal comprising the step of administering at least one CVAC session, said CVAC session having a start point, an end point and more than one target which is executed between said start point and said end point.

8. The method of claim 7, further comprising the step of measuring efficacy of CVAC sessions via changes in physiological markers.

9. The method of claim 8, wherein the physiological marker measured is selected from among:
intervertebral disc hydration;
hematocrit;
erythropoietin (EPO) production;
blood gas composition;
oxygenation of tissues;
angiogenesis within tissues;
blood-perfusion of tissues;
or pain experienced by the patient.

10. The method of claim 7 wherein the user can modulate the parameters of a session.

11. A method of treating inflammation or swelling or a combination thereof in a mammal comprising the step of administering at least one CVAC session, said CVAC session having a start point, an end point and more than one target which is executed between said start point and said end point.

12. The method of claim 11, further comprising the step of measuring efficacy of CVAC sessions via changes in physiological markers.

13. The method of claim 12, wherein the physiological marker measured is selected from among:
hematocrit;
erythropoietin (EPO) production;
blood gas composition;
oxygenation of tissues;
angiogenesis within tissues;
blood-perfusion of tissues;
or extent of tissue necropsy following the onset of inflammation or swelling or combinations thereof.

14. The method of claim 11, further comprising the step of administering least one pharmaceutical compound.

15. The method of claim 11, further comprising the step of administering at least one pharmaceutical compound.

16. The method of claim 11, wherein the user can modulate the parameters of a session.

17. A method of treating a wound in a mammal comprising the step of administering at least one CVAC session, said CVAC session having a start point, an end point and more than one target which is executed between said start point and said end point.

18. The method of claim 17, further comprising the step of measuring efficacy of the at least one CVAC session via changes in physiological markers.

19. The method of claim 18, wherein the physiological marker measured is selected from among:
extent of tissue necropsy following the infliction of a wound;
reduction in healing time of a wound;
tensile strength of the newly grown tissue;
the size of the wound;
hematocrit;
erythropoietin (EPO) production;
blood gas composition;
oxygenation of tissues;
or blood-perfusion of tissues.

20. The method of claim 17, further comprising the step of administering least one non-pharmaceutical therapy.

21. The method of claim 17 wherein the user can modulate the parameters of a session.
